(19) United States
(12) Patent Application Publication
(10) Pub. No.: US 2022/0288303 A1
(43) Pub. Date: Sep. 15, 2022

(54) Diabetes Overlay Patch Shield

(71) Applicants: Andrew Hollis, Davison, MI (US); Emily Hollis, Davison, MI (US)

(72) Inventors: Andrew Hollis, Davison, MI (US); Emily Hollis, Davison, MI (US)

(21) Appl. No.: 17/552,376

(22) Filed: Dec. 16, 2021

Related U.S. Application Data
(63) Continuation of application No. 17/200,790, filed on Mar. 13, 2021, now abandoned.

(51) Int. Cl. A61M 5/142

(52) U.S. Cl. CPC A61M 5/14248; A61M 2230/201

(57) ABSTRACT

An article of manufacture for providing a diabetes overlay patch shield is disclosed. The diabetes overlay patch shield includes a vertical supporting wall structure having an outer surface, a top edge and a bottom edge, the vertical supporting wall structure surrounds a cavity configures to match a shape of an on-body device, a set of supporting fingers coupled to the bottom edge of the vertical supporting wall structure radiating outward from the outer surface, the set of supporting fingers having a bottom surface, and adhesive attached to the bottom surface of the set of supporting fingers to hold the diabetes overlay patch shield. An overlay patch sized to cover the entire the diabetes overlay patch shield is attached over the diabetes overlay patch shield once in place.

### TECHNICAL FIELD

This application relates in general to an article of manufacture for providing diabetes patient aids, and more specifically, to an article of manufacture providing a diabetes overlay patch shield according to the present invention.

### BACKGROUND

Current application of an On-Body Devices (OBDs), that include both a Continuous Glucose Monitor (CGM) and pod insulin pump systems do not utilize very robust adhesive which can lead to premature failure of the adhesive prior to the manufacturer's warranty replacement period. Many of the systems are susceptible to accidental dislodgement. These systems utilize a single overlay patch that exhibit these failures.

Therefore, a need exists for an article of manufacture for providing a diabetes overlay patch shield according to the present invention. The present invention attempts to address the limitations and deficiencies in prior solutions according to the principles and example embodiments disclosed herein.

### SUMMARY

In accordance with the present invention, the above and other problems are solved by providing a diabetes overlay patch shield according to the principles and example embodiments disclosed herein.

In one embodiment, the present invention provides a diabetes overlay patch shield according to the present invention. The diabetes overlay patch shield includes a vertical supporting wall structure having an outer surface, a top edge and a bottom edge, the vertical supporting wall structure surrounds a cavity configures to match a shape of an on-body device, a set of supporting fingers coupled to the bottom edge of the vertical supporting wall structure radiating outward from the outer surface, the set of supporting fingers having a bottom surface, and adhesive attached to the bottom surface of the set of supporting fingers to hold the diabetes overlay patch shield.

In another aspect of the present disclosure, the cavity defined by the vertical supporting wall structure is shaped as a circle.

In another aspect of the present disclosure, the cavity defined by the vertical supporting wall structure is shaped as an oval.

In another aspect of the present disclosure, the cavity defined by the vertical supporting wall structure is a D-shaped cavity.

In another aspect of the present disclosure, an overlay patch sized to cover the entire the diabetes overlay patch shield is attached over the diabetes overlay patch shield once in place.

In another aspect of the present disclosure, the overlay patch has an opening within the center matching the shape of the on-body device within the diabetes overlay patch shield.

In another aspect of the present disclosure, wherein the on-body device is a continuous glucose monitor.

In another aspect of the present disclosure, the on-body device is an insulin pump pod.

In another aspect of the present disclosure, the vertical supporting wall structure has a thickness of 1.2 mm to 2.0 mm and is made of a flexible material such as a Thermoplastic Elastomer TPE or Silicon Elastomer.

In another aspect of the present disclosure, set of fingers have set of fingers have a nominal thickness of 0.75 mm.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIGS. 1a-c illustrate a first embodiment of an article of manufacture providing a diabetes overlay patch shield for an Omnipod™ insulin Pump System according to the present invention.

FIGS. 2a-e illustrate a second example embodiment of an article of manufacture providing a diabetes overlay patch shield for a Dexcom CGM according to the present invention.

FIGS. 3a-e illustrate a third example embodiment of an article of manufacture providing a diabetes overlay patch shield for a Freestyle Libre CGM according to the present invention.

FIG. 4 illustrates top and side views of an article of manufacture providing a diabetes overlay patch shield according to the present invention.

### DETAILED DESCRIPTION

This application relates in general to a system and method for providing a diabetes patient aid, and more specifically, to an article of manufacture for providing a diabetes overlay patch shield according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “patient,” and “user” refer to an entity, e.g., a human, using a diabetes overlay patch shield associated with the invention. The term user herein refers to one or more users.

The term “invention” or “present invention” refers to the invention being applied for via the patent application with the title “Diabetes Overlay Patch Shield.” Invention may be used interchangeably with shield.

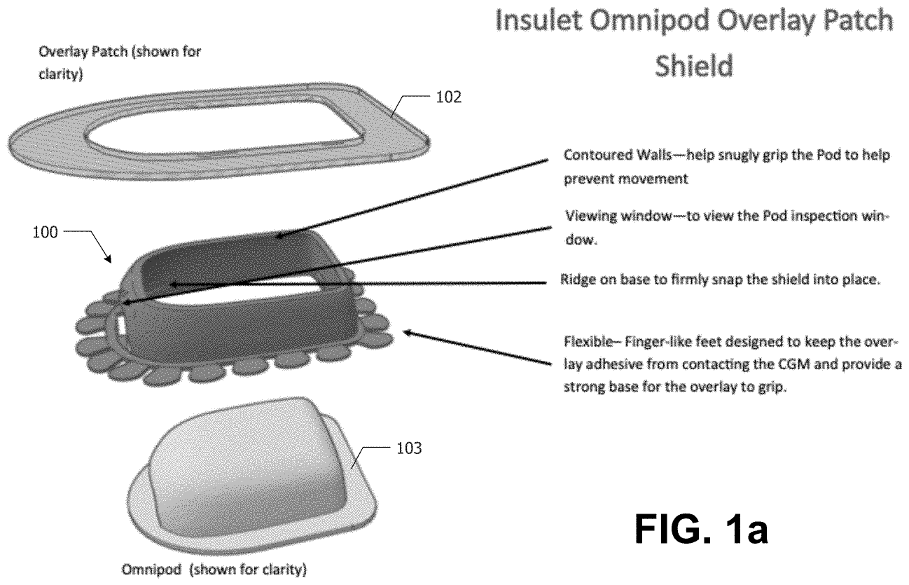

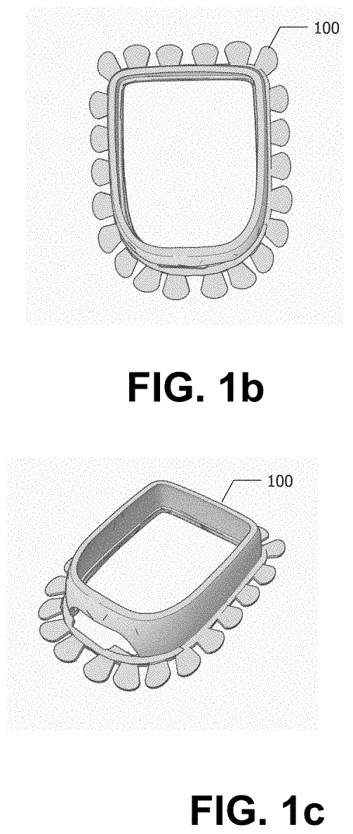

In general, the present disclosure relates to a system and method for providing a diabetes patient aid. To better understand the present invention, FIGS. 1a-c illustrates a first embodiment of an article of manufacture providing a diabetes overlay patch shield for a Omnipod™ OBD insulin pump according to the present invention. FIGS. 1b shows the OmniPod™ 100 top view and FIG. 1c shows the OmniPod™ 100 perspective view.

It is a flexible cover 100 that hugs the contour of the OBD and OmniPod™ Insulin Pump system 102. Around the perimeter of the shield 100 is a flange of fingers 101 that allows an overlay patch 102 to grip the cover and pull it into place. The Omnipod™ cover version 100 has a cutout for easy viewing of an Omnipod™ viewing window that is part of the device. The cover 100 also has a flange 105 around the inside of the base that allows an OmniPod™ Insulin Pump system 102 to clip into place. An overlay patch shield 102 solves two problems: 1. it shields the CGM or pod from contacting the overlay patch adhesive, thereby preventing accidental dislodgement of the system during patch changes, and 2. this shield also grips the body of the OBD or pod and assists in holding it in place to prevent accidental dislodgment from bumps or normal wear (shower, pool, workout etc.).

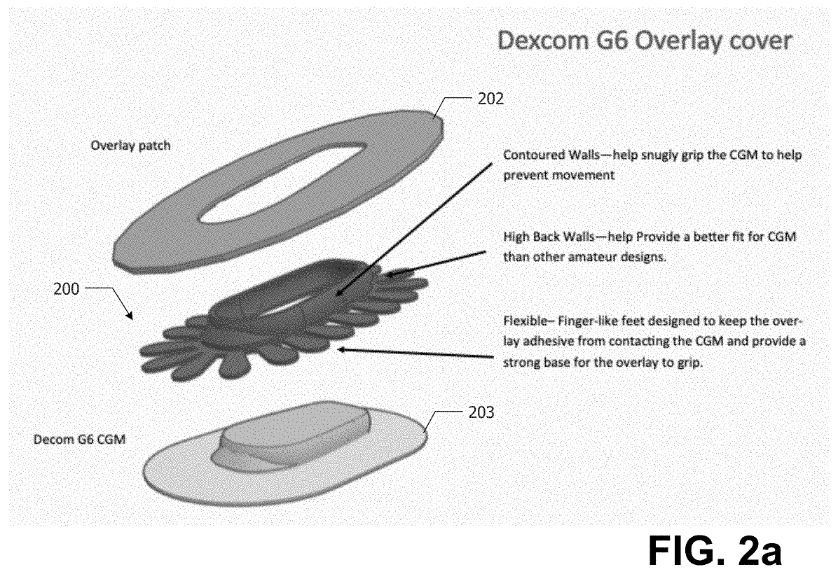

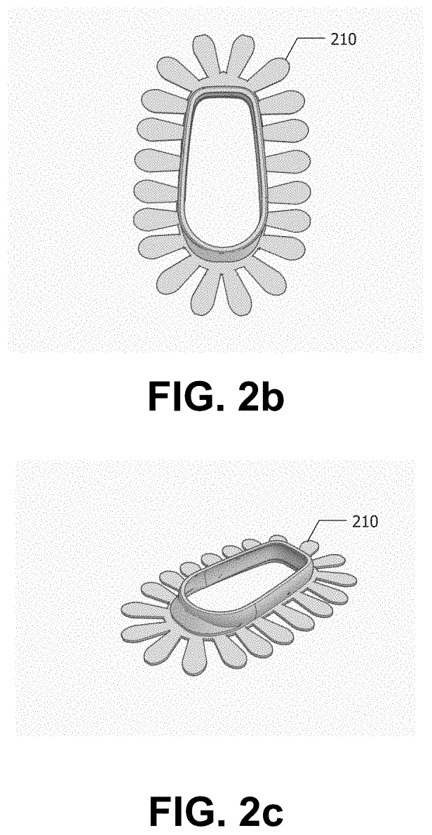

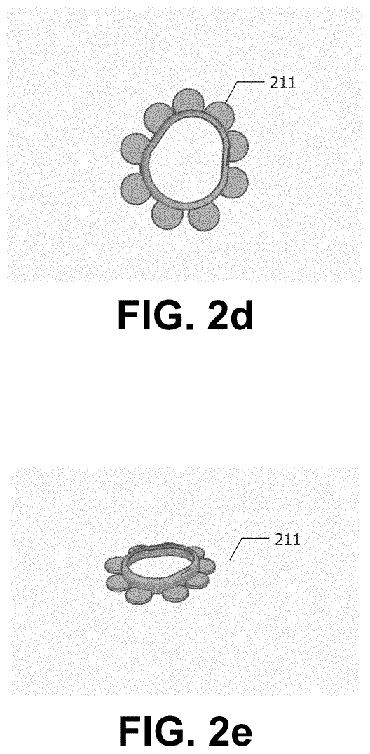

FIGS. 2a-e illustrates a second example embodiment of an article of manufacture providing a diabetes overlay patch shield for a Dexcom™ CGM according to the present invention. It is a flexible cover 200 that hugs the contour of a Dexcom™ CGM 202. Around the perimeter of the shield 200 is a flange of fingers 201 that allows an overlay patch 202 to grip the cover and pull it into place.

The second embodiment 200 utilizes an oval-shaped flexible cover 200 shaped to match the outside perimeter of the Dexcom™ CGM 202. The contoured walls 205 of the oval-shaped flexible cover 200 assists in snugly gripping the Dexcom™ CGM 202 to prevent its movement while being worn by a patient. These contoured walls 202 also provide a high-back wall structure providing a secure fit for the Dexcom™ CGM 202.

FIGS. 2b shows the Dexcom™ CGM 210 top view and FIG. 2c shows the Dexcom™ CGM 210 perspective view. FIGS. 2d shows the Dexcom™ CGM 211 top view and FIG. 2e shows the Dexcom™ CGM 211 perspective view.

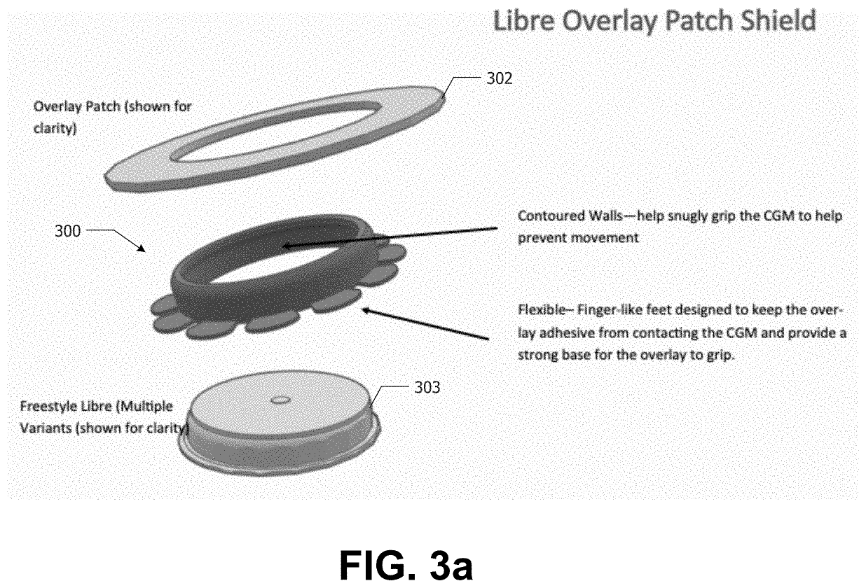

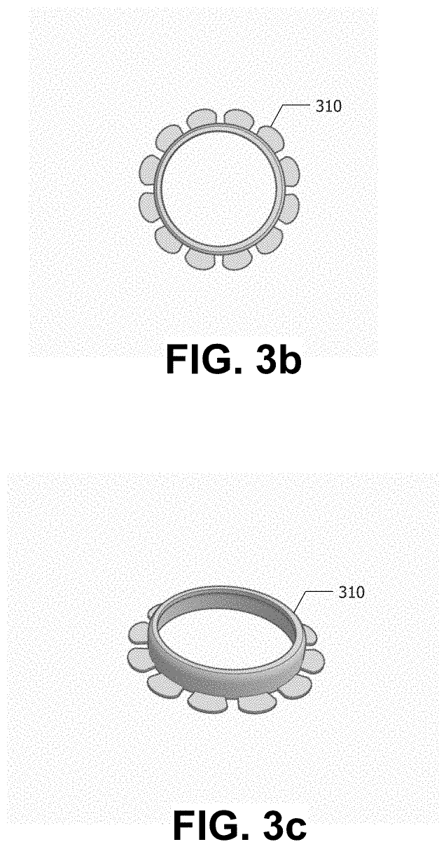

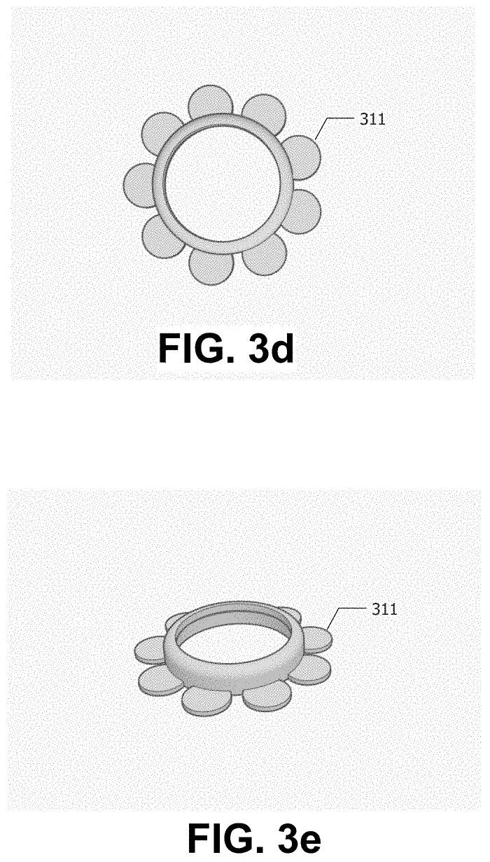

FIGS. 3a-e illustrates a third example embodiment of an article of manufacture providing a diabetes overlay patch shield for a Freestyle Libre™ CGM according to the present invention. The third embodiment includes a flexible cover 300 that hugs the contour of the Freestyle Libre™ CGM 302. Around the perimeter of the shield 300 is a flange of fingers 301 that allows an overlay patch 302 to grip the cover and pull it into place.

The third embodiment 300 utilizes a round-shaped flexible cover 300 shaped to match the outside perimeter of the Freestyle Libre™ COM 302. The contoured walls 305 of the oval-shaped flexible cover 300 assist in snugly gripping the Freestyle Libre™ COM 302 to prevent its dislodgement while being worn by a patient.

FIGS. 3b shows the Freestyle Libre™ 1-2 CGM 310 top view and FIG. 3c shows the Freestyle Libre™ 1-2 CGM 310 perspective view. FIGS. 3d shows the Freestyle Libre™ 3 COM 311 top view and FIG. 3e shows the Freestyle Libre™ 3 CGM 311 perspective view.

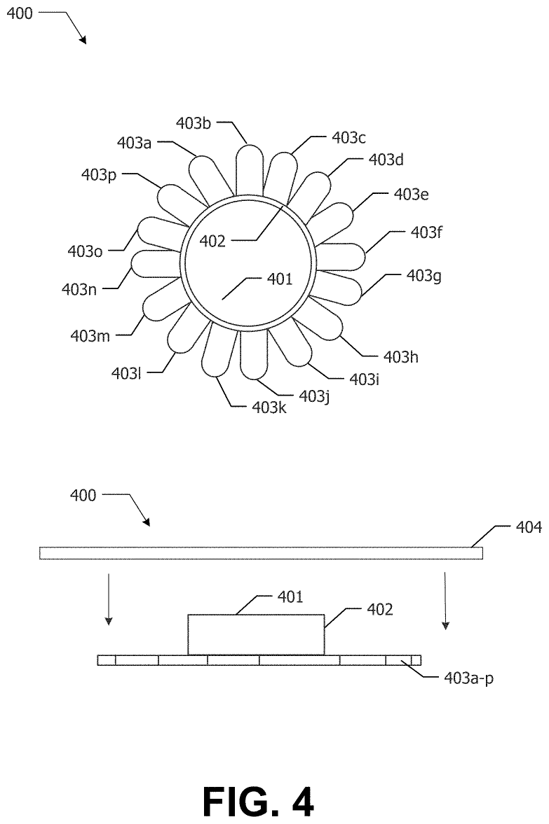

FIG. 4 illustrates top and side views of an article of manufacture providing a diabetes overlay patch shield according to the present invention. The above embodiments of the diabetes overlay patch shield 400 comprises an inner cavity 401 for accepting a CGM and OBD devices that is surrounding by a supporting wall structure 402. A set of fingers 403a-p are coupled to an outer surface of the supporting wall structure 402 and radiating outward to rest upon the skin of a patient using the CGM and/or OBD. There are no preferred set of fingers as the diabetes overlay patch shield 400 is tailored to fit the needs of the various devices. The number of fingers 403a-p and spacing will be determined by the area of the OBD adhesive. There needs to be enough fingers equally spaced to adequately cover the adhesive to prevent aftermarket overlay patches from contacting the adhesive. The length of the fingers needs to be such that they completely overlap the OBD adhesive but must be short enough for the aftermarket overlay patches to adhere to the wearer's skin.

The overlay patches 404 are a separate product that users can purchase from a retailer of their choice. These patches 404 typically come in standard sizes. They typically have the hole for the OBD cutout already.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. An article of manufacture for providing a diabetes overlay patch shield, the diabetes overlay patch shield comprises:
a vertical supporting wall structure having an outer surface, a top edge and a bottom edge, the vertical supporting wall structure surrounds a cavity configures to match a shape of an on-body device;
a set of supporting fingers coupled to the bottom edge of the vertical supporting wall structure radiating outward from the outer surface, the set of supporting fingers having a bottom surface; and
adhesive attached to the bottom surface of the set of supporting fingers to hold the diabetes overlay patch shield.

2. The article of manufacture according to claim 1, wherein the cavity defined by the vertical supporting wall structure is shaped as a circle.

3. The article of manufacture according to claim 1, wherein the cavity defined by the vertical supporting wall structure is shaped as an oval.

4. The article of manufacture according to claim 1, wherein the cavity defined by the vertical supporting wall structure is a D-shaped cavity.

5. The article of manufacture according to claim 1, wherein an overlay patch sized to cover the entire the diabetes overlay patch shield is attached over the diabetes overlay patch shield once in place.

6. The article of manufacture according to claim 5, wherein the overlay patch has an opening within the center matching the shape of the on-body device within the diabetes overlay patch shield.

7. The article of manufacture according to claim 6, wherein the on-body device is a continuous glucose monitor.

8. The article of manufacture according to claim 6, wherein the on-body device is an insulin pump pod.

9. The article of manufacture according to claim 1, wherein the vertical supporting wall structure has a thickness of 1.2 mm to 2.0 mm and is made of a flexible material such as a Thermoplastic Elastomer TPE or Silicon Elastomer.

10. The article of manufacture according to claim 1, wherein the set of fingers have set of fingers have a nominal thickness of 0.75 mm.
